(19) United States
(12) Patent Application Publication
(10) Pub. No.: US 2016/0369313 A1
(43) Pub. Date: Dec. 22, 2016

(54) GALACTO-OLIGOSACCHARIDE-CONTAINING COMPOSITION AND A METHOD OF PRODUCING IT

(71) Applicant: ARLA FOODS AMBA, Viby (DK)

(72) Inventors: Hans Bertelsen, Videbaek (DK); Peter Langborg Wejse, Aarhus N (DK)

(21) Appl. No.: 15/217,266

(22) Filed: Jul. 22, 2016

Related U.S. Application Data
(63) Continuation of application No. 13/811,171, filed on Mar. 8, 2013, now abandoned.
(60) Provisional application No. 61/365,560, filed on Jul. 19, 2010.

(30) Foreign Application Priority Data
Jul. 19, 2010 (EP) 10169981.7

(51) Int. Cl. C12P 19/12; A23L 33/00; C07H 3/06; A23L 33/21; C12P 19/14; C12N 9/38

(52) U.S. Cl. CPC C12P 19/12; C12P 19/14; C12N 9/2471; C12Y 302/01023; C07H 3/06; A23L 33/21; A23L 33/40; A23V 2002/00

(57) ABSTRACT

The present invention relates to a method of producing compositions containing galacto-oligosaccharides as well to galacto-oligosaccharide-containing compositions as such.

### CROSS REFERENCE

This is a continuation of U.S. application Ser. No. 13/811,171 filed Mar. 8, 2013, which is a U.S. National Phase Application of PCT/EP2011/062355 filed Jul. 19, 2011, which claims priority to U.S. Provisional Application No. 61/365,560 filed Jul. 19, 2010 and European Application No. EP10169981.7 filed Jul. 19, 2010, each of which is hereby incorporated by reference in their entirety.

### FIELD OF THE INVENTION

The present invention relates to a galacto-oligosaccharide-containing composition as well as an efficient method of producing it.

### BACKGROUND

Human breast milk is known to contain a number of different oligosaccharides which are ascribed some of the beneficial health effects of breast feeding infants (Kunz et al. (2000)). For example, some oligosaccharides, such as FOS, GOS, or inulin, are so-called prebiotics, which means that they promote the beneficial bacteria of the gastrointestinal system and disfavour the harmful bacteria. Oligosaccharides are, due to their health promoting effects, frequently used in functional food products, such as infant formulas and clinical nutrition.

There are several approaches to the production of oligosaccharides. One approach is based on isolating oligosaccharides from naturally occurring sources. Fructose-oligosaccharide (FOS) and inulin are for example found naturally in Jerusalem artichoke, burdock, chicory, leeks, onions and asparagus and may be isolated from these crops. Preparation of inulin from chicory roots is e.g. described in Frank (2002). This approach to the production of oligosaccharides is limited by the 25 availability of suitable crops and may be impossible to implement for more complex oligosaccharides.

Another approach is based on enzymatic synthesis in which enzymes catalyse the synthesis of the oligosaccharides. Yun (1996) describes the enzymatic production of fructo-oligosaccharides using enzymes having fructosyltransferase activity and using sucrose as substrate for the enzyme. Another example of enzymatic synthesis is described in WO 01/90,317 A2 which discloses a method of producing galacto-oligosaccharides (GOS) of the formula Gal-Gal-Glc using a special beta-galactosidase enzyme and lactose as substrate.

### SUMMARY OF THE INVENTION

An object of the invention is to provide improved methods of producing galacto-oligosaccharides. It is furthermore an object of the invention to provide improved compositions containing galacto-oligosaccharides.

The present inventors have observed that, surprisingly, enzymes having beta-galactosidase activity, and preferably having a T-value of at most 0.9, can be used for highly effective synthesis of a special type of galacto-oligosaccharides, in which the galactosyl acceptor is different from the galactosyl donor.

Thus, an aspect of the invention relates to a method of producing a composition comprising one or more galacto-oligosaccharides, the method comprising the steps of:

a) providing a mixture comprising
a galactosyl donor comprising a galactosyl group bound to a leaving group,
a galactosyl acceptor which is different from the galactosyl donor, and
   wherein the molar ratio between the galactosyl acceptor and the galactosyl donor is at least 1:10, and wherein the mixture comprises at least 0.05 mol/L of the galactosyl acceptor,

b) providing an enzyme having beta-galactosidase activity, said enzyme contacting the mixture,

c) allowing the enzyme to release the leaving group of the galactosyl donor and transfer the galactosyl group of the galactosyl donor to the galactosyl acceptor, thus forming the galacto-oligosaccharide, and thereby obtaining the composition comprising the one or more galacto-oligosaccharide(s).

This invention opens up for cheap and efficient production of complex galacto-oligosaccharide compositions in high yield. The present invention furthermore appears to reduce the degree of self-galactosylation of the galactosyl donor, which may result in undesired by-products, which are expensive to remove from the composition.

Preferably, the enzyme has transgalactosylation activity in addition to beta-galactosidase activity. It may also be preferred that the enzyme has a T-value of at most 0.9.

In the context of the present invention the term “transgalactosylation activity” of a beta-galactosidase enzyme relates to the ability of the enzyme to transfer a galactosyl group from a donor molecule, e.g. a lactose molecule, to a non-water molecule, e.g. another lactose molecule.

The T-value is a measure of the transgalactosylation efficiency of a beta-galactosidase enzyme using lactose both as galactosyl donor and acceptor. The determination of the T-value of a beta-galactosidase enzyme is performed according to the assay and the formula described in Example 2. The T-value is calculated using the formula:

$$T \text{-} value = \frac{amount of produced galactose ( in mol )}{amount of used lactose ( in mol )}$$

A lactase enzyme without any transgalactosylation activity will produce one mol galactose for each used mol lactose and would have a T-value of 1. A beta-galactosidase having an extremely high transgalactosylation activity would use nearly all the galactosyl groups from the lactose for transgalactosylation instead of generating galactose, and would consequently have a T-value near 0.

Yet an aspect of the invention relates to a composition comprising one or more galacto-oligosaccharide(s), which composition is obtainable by the method as described herein.

Additional objects and advantages of the invention are described below.

### BRIEF DESCRIPTION OF THE FIGURES

FIG. 1a shows a HPLC chromatogram of the mixture described in Example 3 (containing lactose and L-fucose) before incubation with the enzyme.

FIG. 1b shows a HPLC chromatogram of the mixture of Example 3 after incubation with the enzyme, where peaks of L-fucose-containing galacto-oligosaccharides (peaks 6, 7 and 8) are clearly present.

FIG. 2 contains a plot of the concentration (arbitrary units) of lactose, glucose and galactose of the mixture of Example 3 during the enzymatic reaction.

FIG. 3 contains a plot of the concentration (arbitrary units) of the oligosaccharides Gal-Fuc, Gal-Gal-Fuc/Gal-Gal-Glc, and Gal-Gal-Gal-Fuc/Gal-Gal-Gal-Glc of the mixture of Example 3 during the enzymatic reaction.

FIG. 4 contains a plot of the concentration (arbitrary units) of the oligosaccharides Gal-Fuc, Gal-Gal-Fuc/Gal-Gal-Glc, and Gal-Gal-Gal-Fuc/Gal-Gal-Gal-Glc of the mixture of Example 4 during the enzymatic reaction.

FIG. 5 contains a plot of the concentration (arbitrary units) of the oligosaccharides Gal-GaINAc, Gal-Gal- GaINAc/Gal-Gal-Glc, and Gal-Gal-Gal-GaINAc/Gal-Gal-Gal-Glc of the mixture of Example 5 during the enzymatic reaction.

FIG. 6 contains a plot of the concentration (arbitrary units) of the oligosaccharides Gal-Xyl, Gal-Gal- Xyl/Gal-Gal-Glc, and Gal-Gal-Gal- Xyl/Gal-Gal-Gal-Glc of the mixture of Example 6 during the enzymatic reaction.

### DETAILED DESCRIPTION OF THE INVENTION

As mentioned, an aspect of the invention relates to a method of producing a composition comprising one or more galacto-oligosaccharide(s), the method comprising the steps of:

a) providing a mixture comprising
a galactosyl donor comprising a galactosyl group bound to a leaving group,
a galactosyl acceptor which is different from the galactosyl donor, and
   wherein the molar ratio between the galactosyl acceptor and the galactosyl donor is at least 1:10, and wherein the mixture comprises at least 0.05 mol/L of the galactosyl acceptor,

b) providing an enzyme having beta-galactosidase activity and preferably having a T-value of at most 0.9, said enzyme contacting the mixture, and

c) allowing the enzyme to release the leaving group of the galactosyl donor and transfer the galactosyl group of the galactosyl donor to the galactosyl acceptor, thus forming the galacto-oligosaccharide, and thereby obtaining the composition comprising the one or more galacto-oligosaccharide(s).

In the context of the present invention, the term “glycosyl group” relates to a group obtained by removing one or two hydroxyl groups from a monosaccharide or a lower oligosaccharide, such as a di- or tri-saccharide, or from corresponding sugar-alcohols. The term is used herein to describe various building blocks of galactosyl donors, galactosyl acceptors and oligosaccharides.

The abbreviations of the most common saccharides and their corresponding glycosyl groups are shown below.

| Saccharide | Abbreviation | Name of glycosyl group |
|---|---|---|
| glucose | Glc | glucosyl |
| galactose | Gal | galactosyl |
| fucose | Fuc | fucosyl |
| mannose | Man | mannosyl |
| xylose | Xyl | xylosyl |
| N-acetylgalactosamine | GalNAc | N-acetylgalactosaminyl |
| Lactose | Lac | lactosyl |

In the context of the present invention, the term “oligosaccharide” relates to a molecule comprising at least two glycosyl groups, and preferably at least three, which may be different or the same type. The at least two glycosyl groups are preferably bound via an O-glycosylic bond. An oligosaccharide may be a linear chain of glycosyl groups or it may have a branched structure. Oligosaccharides may e.g. be represented as a stoichiometric formula, e.g. (Gal)₃Glc, or as general formulas, e.g. Gal-Gal-Gal-Glc, Gal-Gal-Glc-Gal, or Gal-(Gal-)Glc-Gal. The stoichiometric formulas provide information regarding which glycosyl groups an oligosaccharide, or a group of oligosaccharides, contains, but not the relative position of these, whereas the general formulas also contain general information regarding the relative positions of the glycosyl groups.

In the context of the present invention the term “homo-oligosaccharide” relates to an oligosaccharide containing only one type of glycosyl group. Examples of homo-oligosaccharides are Gal-Gal-Gal-Gal and Glc-Glc-Glc.

In the context of the present invention the term “hetero-oligosaccharide” relates to an oligosaccharide which contains different glycosyl groups, e.g. Gal-Gal-Glc, or Gal-Gal-Fuc.

In the context of the present invention, the prefix “galacto-” used together with the term “oligosaccharide” indicates that the oligosaccharide contains galactosyl groups as the repeating unit. The “homo-” or “hetero-” prefix may be used together with the “galacto-” prefix. Both Gal-Gal-Glc and Gal-Gal-Gal-Gal are galacto-oligosaccharides. Gal-Gal-Glc is a hetero-galacto-oligosaccharide and Gal-Gal-Gal-Gal is a homo-galacto-oligosaccharide.

In the context of the present invention, “X” represents a galactosyl acceptor as defined herein. “—X” represents the glycosyl group corresponding to the galactosyl acceptor, and particularly the glycosyl group bound to another group. “—” symbolises the bond. The glycosyl group is preferably bound via the 3-, 4-, 5- or 6-position of the glycosyl group, and preferably via an O-glycosylic bond. In the context of the present invention, “Gal-” represents a galactosyl group bound to another group, preferably via the 1-position of the galactosyl group, and preferably via an O-glycosylic bond.

In the context of the present invention, “-Gal-” represents a galactosyl group bound to two other groups. The left bond is preferably made via the 4- or 6-position of the galactosyl group, and preferably via an O-glycosylic bond. The right bond is preferably made via the 1-position of the galactosyl group, and preferably via an O-glycosylic bond.

Bonds between two galactosyl groups are typically 1-4 or 1-6 bonds, and normally O-glycosylic bonds. A bond between a galactosyl group and a nitrogen-containing acceptor may alternatively be an N-glycosylic bond.

In the context of the present invention the terms “method” and “process” are used interchangeably.

Step a) involves the provision of the mixture in which the oligosaccharides are to be produced.

The mixture is preferably a liquid mixture and may e.g. be an aqueous solution containing the galactosyl acceptor and the galactosyl donor.

In some embodiments of the invention the molar ratio between the galactosyl acceptor and the galactosyl donor of the mixture of step a) is at least 1:5, preferably at least 1:1, and even more preferably at least 5:1. For example, the molar ratio between the galactosyl acceptor and the galactosyl donor of the mixture of step a) may be at least 10:1, such as at least 15:1.

The molar ratio between the galactosyl acceptor and the galactosyl donor of the mixture of step a) may e.g. be in the range of 1:10-100:1.

In some embodiments of the invention the molar ratio between the galactosyl acceptor and the galactosyl donor of the mixture of step a) is in the range of 1:10-50:1, preferably in the range of 1:5-30:1, and even more preferably in the range of 1:1-20:1. For example, the molar ratio between the galactosyl acceptor and the galactosyl donor of the mixture of step a) may e.g. be in the range of 2:1-40:1, preferably in the range of 4:1-30:1, and even more preferably in the range of 10:1-25:1.

As mentioned, the galactosyl donors contain a galactosyl group covalently bound to a leaving group. The galactosyl group is preferably a β-D-galactopyranosyl group. Furthermore, the galactosyl group is preferably bound to the leaving group via an O-glycosidic bond from the 1-position of the galactosyl group.

The leaving group of the galactosyl donor may for example be a glycosyl group and/or a sugar-alcohol group. If the leaving group is a glycosyl group of a mono- or disaccharide or a corresponding sugar-alcohol, the galactosyl group is preferably bound to the leaving group via an O-glycosidic bond from the 1-position of the galactosyl group, which bond attaches to the 4-position of a monosaccharide-type leaving group or to the 4′-position of a disaccharide-type leaving group.

In the context of the present invention, the phrase “Y and/or X” means “Y” or “X” or “Y and X”. Along the same line of logic, the phrase “X₁, X₂, . . . , Xᵢ₋₁, and/or Xᵢ” means “X₁” or “X₂” or . . . or “Xᵢ₋₁” or “Xᵢ” or any combination of the components: X₁, X₂, . . . Xᵢ₋₁, and Xᵢ.

In some embodiments of the invention the galactosyl donor has a molar weight of at most 1000 g/mol. For example, the galactosyl donor may have a molar weight of at most 500 g/mol. It may even be preferred that the galactosyl donor has a molar weight of at most 350 g/mol.

Disaccharides are a presently preferred type of galactosyl donor. Alternatively, or additionally, tri-saccharides may be used as galactosyl donors as well. Thus, it is envisioned that the mixture may contain a combination of different galactosyl donors.

In some preferred embodiments of the invention the galactosyl donor is lactose. Another example of a useful galactosyl donor is lactulose. Yet an example of a useful galactosyl donor is lactitol.

In the context of the present invention the term “lactose” relates to the disaccharide β-D-galactopyranosyl-(1→4)-D-glucose, which is also referred to as milk sugar, and which is the most predominant saccharide of bovine milk.

The galactosyl donor may be provided via any useful galactosyl donor source, both industrially refined sources, such as purified lactose, and/or natural sources, such as whey permeate, i.e. deproteinated whey prepared by ultrafiltration of whey.

The galactosyl acceptor may be any molecule capable of accepting a galactosyl group from the enzyme and typically contains hydroxyl groups, and preferably alcoholic hydroxyl groups. The term “accepting” means that the galactosyl group of the donor should be covalently bound to the acceptor, e.g. via an O-glycosylic bond.

In some embodiments of the invention the galactosyl acceptor comprises one or more alcoholic hydroxyl group(s). For example, the galactosyl acceptor may be a polyol.

In the context of the present invention the term “polyol” relates to a molecule comprising at least two alcoholic hydroxyl groups.

In some preferred embodiments of the invention the galactosyl acceptor is not lactose. It may furthermore be preferred that the galactosyl acceptor is not glucose.

In some preferred embodiments of the invention the galactosyl acceptor is different from the galactosyl donor. It is particularly preferred to use a relatively cheap galactosyl donor, such as lactose, as galactosyl source and a biologically interesting acceptor, such as fucose, as galactosyl acceptor.

In some embodiments of the invention the galactosyl acceptor is not lactose, galactose, or glucose.

In some embodiments of the invention the galactosyl acceptor is not glucose or oligosaccharides of the general formula Gal-(Gal)ᵢ-Glc, where i is a non-negative integer, i.e. for example 0, 1, 2, 3, or 4.

In some embodiments of the invention the galactosyl acceptor is not galactose or oligosaccharides of the general formula Gal-(Gal)ᵢ-Gal, where i is a non-negative integer.

Galactosyl acceptors having various molar weights may be used, but galactosyl acceptors having a molar weight of at least 100 g/mol are presently preferred.

In some embodiments of the invention the galactosyl acceptor has a molar weight of at most 1000 g/mol. For example, the galactosyl acceptor may have a molar weight of at most 500 g/mol. It may even be preferred that the galactosyl acceptor has a molar weight of at most 350 g/mol. The galactosyl acceptor may for example have a molar weight of at most 200 g/mol.

In some preferred embodiments of the invention the galactosyl acceptor is a saccharide. The galactosyl acceptor may for example be a mono-saccharide. Alternatively, the galactosyl acceptor may be a di-saccharide.

For example, the galactosyl acceptor may be a pentose. The galactosyl acceptor may e.g. be arabinose. Another example of a useful pentose is xylose. Yet an example of a useful pentose is ribose. The galactosyl acceptor may for example be a pentose selected from the group consisting of arabinose, xylose, and ribose.

Hexoses are another group of useful galactosyl acceptors. The galactosyl acceptor may e.g. be mannose. Another example of a useful hexose is galactose. Yet an example of a useful hexose is tagatose. A further example of a useful hexose is fructose. The galactosyl acceptor may for example be a hexose selected from the group consisting of mannose, galactose, tagatose, and fructose.

In some preferred embodiments of the invention the galactosyl acceptor is a deoxy-hexose. The galactosyl acceptor may for example be fucose, such as e.g. D-fucose, L-fucose, or a mixture thereof.

Alternatively, the galactosyl acceptor may be an oligosaccharide, such as e.g. a di-saccharide or a tri-saccharide. An example of a useful di-saccharide is maltose. Another example of a useful di-saccharide is lactulose.

Yet a useful group of galactosyl acceptors is saccharide derivatives.

In the context of the present invention the term “saccharide derivative” pertains to a saccharide containing one or more non-hydroxyl functional group(s). Examples of such functional groups are a carboxyl group, an amino group, an N-acetylamino group and/or a thiol group. Saccharides which contain an aldehyde group at the 1-position or a ketone group at the 2-position are not considered saccharide derivatives as such unless the saccharides comprise some of the non-hydroxyl functional groups mentioned above.

An example of a useful saccharide derivative is N-acetyl galactosamine. Another example of a useful saccharide derivative is sialic acid. Yet an example of a useful saccharide derivative is sialyl lactose. Thus, the galactosyl acceptor may be a saccharide derivative selected from the group consisting of N-acetyl galactosamine, sialic acid, and sialyl lactose.

Another group of useful galactosyl acceptors is sugar alcohols. Thus, in some embodiments of the invention the galactosyl acceptor is a sugar alcohol. Examples of useful sugar alcohols are sorbitol, xylitol, lactitol, and/or maltitol.

Contrary to the above-mentioned galactosyl acceptors, the present inventors have found that N-acetyl glucosamine and glucose are less efficient galactosyl acceptors. Thus, in some embodiments of the invention the galactosyl acceptor is not glucose or N-acetyl glucosamine.

The mixture may contain one or more further galactosyl acceptor(s) different from the first type of galactosyl acceptor. The different types of galactosyl acceptors of the mixture may e.g. be selected among the galactosyl acceptor types mentioned herein.

In some preferred embodiments of the invention the produced galactosylated acceptors act as a new type of galactosyl acceptor and can be galactosylated as well. In this way, galacto-oligosaccharides may be produced which have the stoichiometric formula Galᵢ₊₁X, where i is a non-negative integer. Normally, the most predominant species are GalX, Gal₂X, and Gal₃X.

In other preferred embodiments of the invention the produced galactosylated acceptors act as a new type of galactosyl acceptor and can be galactosylated as well. In this way, galacto-oligosaccharides may be produced which have the general formula Gal-(Gal)ᵢ-X, where i is a non-negative integer. Normally, the most predominant species are Gal-X, Gal-Gal-X, and Gal-Gal-Gal-X.

In some embodiments of the invention the mixture of step a) comprises the galactosyl donor in a concentration of at most 0.7 mol/L, preferably at most 0.4 mol/L, and even more preferably at most 0.2 mol/L. The mixture may e.g. comprise the galactosyl donor in a concentration in the range of 0.001-0.7 mol/L, preferably in the range of 0.01-0.5 mol/L, and even more preferred in the range of 0.02-0.2 mol/L.

Alternatively, the mixture of step a) may comprise the galactosyl donor in a concentration of at most 0.3 mol/L, preferably at most 0.1 mol/L, and even more preferably at most 0.05 mol/L. The mixture may e.g. comprise the galactosyl donor in a concentration in the range of 0.001-0.2 mol/L, preferably in the range of 0.005-0.1 mol/L, and even more preferred in the range of 0.01-0.05 mol/L.

It should be noted that galactosylated galactosyl acceptor and galactosylated galactosyl donor may to a limited extent act as a galactosyl donor, but galactosylated galactosyl acceptor and galactosylated galactosyl donor are not considered a galactosyl donor in the context of the present invention and do not contribute to the concentrations or ratios of galactosyl donor mentioned herein.

The galactosyl acceptor may be used in a range of difference concentrations. It is, however, preferred to avoid saturating the mixture with the galactosyl acceptor since excess galactosyl acceptor normally has to be removed from the galacto-oligosaccharide-containing composition of the invention.

In some embodiments of the invention the mixture of step a) comprises the galactosyl acceptor in an amount of at least 0.05 mol/L, preferably at least 0.10 mol/L, and even more preferably at least 0.30 mol/L. Even higher concentrations of the galactosyl acceptor may be preferred, thus the mixture of step a) may e.g. comprise the galactosyl acceptor in an amount of at least 0.5 mol/L, preferably at least 0.7 mol/L, and even more preferably at least 1 mol/L.

The mixture may e.g. comprise the galactosyl acceptor in a concentration in the range of 0.05 mol/L-5 mol/L, preferably in the range of 0.1 mol/L-2 mol/L, and even more preferably in the range of 0.3 mol/L-1 mol/L.

However, in some embodiments a relatively low concentration of the galactosyl acceptor is preferred in which case the mixture may e.g. comprise the galactosyl acceptor in a concentration of at most 2 mol/L, preferably at most 0.5 mol/L, and even more preferably at most 0.2 mol/L. For example, the mixture may comprise the galactosyl acceptor in a concentration in the range of 0.05 mol/L-2 mol/L, preferably in the range of 0.06 mol/L-1 mol/L, and even more preferably in the range of 0.08 mol/L-0.8 mol/L.

In addition to galactosyl acceptor and galactosyl donor, the mixture may furthermore contain various additives for optimizing the conditions for the enzymatic reaction.

The mixture may for example contain one or more pH buffer(s) for adjusting the pH of the mixture to the pH optimum of the enzyme. Alternatively, or in addition, the mixture may comprise water soluble salts containing one or more metal ions. Depending on the specific enzyme, metal ions such as Ca²⁺, Zn²⁺, or Mg²⁺ may e.g. be used. Note, however, that some enzymes are insensitive to the presence of metal ions in the mixture.

Conventional methods of synthesising oligosaccharides often employ water-activity-lowering agents, such as e.g. glycerol, ethylene glycol, propylene glycol, polyethyleneglycol (PEG). The present invention advantageously makes it possible to perform efficient synthesis of galacto-oligosaccharides without the use of such water-activity-lowering agents. Thus, in some preferred embodiments of the invention the mixture contains water-activity-lowering agent in an amount of at most 5% by weight relative to the weight of the mixture, preferably at most 1% by weight, and even more preferably at most 0.1% by weight. For example, the mixture may contain water-activity-lowering agent in an amount of at most 0.05% by weight relative to the weight of the mixture.

The mixture of step a) or the ingredients forming the mixture may have been heat treated before the reaction with enzyme to avoid microbial growth during the reaction. The usual heat treatment processes, such as pasteurisation (e.g. 72 degrees C. for 15 seconds), high pasteurisation (e.g. 90 degrees C. for 15 seconds), or UHT treatment (e.g. 140 degrees C. for 4 seconds), may be used. Care should be taken when heat treating temperature labile enzymes.

Step b) involves the provision of an enzyme, which preferably has beta-galactosidase activity, and preferably a T-value of at most 0.9. It should be noted that the method may furthermore involve the use of additional enzymes, e.g. enzymes having a different enzymatic activity than beta-galactosidase activity or transgalactosylation activity.

In the context of the present invention the term “beta-galactosidase activity” relates to enzymatic catalysis of the hydrolysis of terminal non-reducing β-D-galactose residues in β-D-galactosides, such as lactose. The enzyme used in the invention preferably belongs to the class EC 3.2.1.23.

In some embodiments of the invention, the T-value of the enzyme is at most 0.8, preferably at most 0.7, and even more preferably at most 0.6. For example, the T-value of the enzyme may be at most 0.5. Preferably the T-value of the enzyme may be at most 0.4. It may even be more preferred that the T-value of the enzyme is at most 0.3.

Even lower T-values may be preferred, such as at most 0.2.

Useful enzymes may e.g. be derived from a peptide encoded by the DNA sequence of SEQ ID NO. 1. An example of such a peptide from which useful enzymes may e.g. be derived is the peptide having amino acid sequence of SEQ ID NO. 2.

SEQ ID NO. 1 and SEQ ID NO. 2 can be found in the PCT application WO 01/90,317 A2, where they are referred to as SEQ ID NO: 1 and SEQ ID NO: 2. Additionally, further useful enzymes may be also be found in WO 01/90,317 A2.

In some preferred embodiments of the invention the enzyme comprises an amino acid sequence having a sequence identity of at least 80% relative to the peptide of SEQ ID NO. 2. For example, the enzyme may comprise an amino acid sequence having a sequence identity of at least 90% relative to the peptide of SEQ ID NO. 2, preferably at least 95%, and even more preferably at least 97.5%. In some instances the enzyme may comprise an amino acid sequence having a sequence identity of at least 99% relative to the peptide of SEQ ID NO. 2.

In the context of the present invention the term “sequence identity” relates to a quantitative measure of the degree of identity between two amino acid sequences of equal length or between two nucleic acid sequences of equal length. If the two sequences to be compared are not of equal length, they must be aligned to the best possible fit. The sequence identity can be calculated as

N_{ref}−N_{dif})*100)/(N_{ref}),

wherein N_{dif} is the total number of non-identical residues in the two sequences when aligned, and wherein N_{ref} is the number of residues in one of the sequences. Hence, the DNA sequence AGTCAGTC will have a sequence identity of 75% with the sequence AATCAATC (N_{diff}=2 and N_{ref}=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can for example be calculated using appropriate BLAST-programs, such as the BLAST p-algorithm provided by National Center for Biotechnology Information (NCBI), USA.

In other preferred embodiments of the invention the amino acid sequence of the enzyme has a sequence identity of at least 80% relative to the peptide of SEQ ID NO. 2. For example, the amino acid sequence of the enzyme may have a sequence identity of at least 90% relative to the peptide of SEQ ID NO. 2, preferably at least 95%, and even more preferably at least 97.5%. In some instances the amino acid sequence of the enzyme may have a sequence identity of at least 99% relative to the peptide of SEQ ID NO. 2.

In some preferred embodiments of the invention the enzyme comprises an amino acid sequence having a sequence identity of at least 80% relative to the amino acid sequence Met (1) to Gly (1752) of SEQ ID NO. 2. For example, the enzyme may comprise an amino acid sequence having a sequence identity of at least 90% relative to the amino acid sequence Met (1) to Gly (1752) of SEQ ID NO. 2, preferably at least 95%, and even more preferably at least 97.5%. In some instances the enzyme may comprise an amino acid sequence having a sequence identity of at least 99% relative to the amino acid sequence Met (1) to Gly (1752) of SEQ ID NO. 2.

In other preferred embodiments of the invention the amino acid sequence of the enzyme has a sequence identity of at least 80% relative to the amino acid sequence Met (1) to Gly (1752) of SEQ ID NO. 2. For example, the amino acid sequence of the enzyme may have a sequence identity of at least 90% relative to the amino acid sequence Met (1) to Gly (1752) of SEQ ID NO. 2, preferably at least 95%, and even more preferably at least 97.5%. In some instances the amino acid sequence of the enzyme may have a sequence identity of at least 99% relative to the amino acid sequence Met (1) to Gly (1752) of SEQ ID NO. 2. Thus, the enzyme may e.g. have the amino acid sequence Met (1) to Gly (1752) of SEQ ID NO. 2.

In some preferred embodiments of the invention the enzyme comprises an amino acid sequence having a sequence identity of at least 80% relative to the amino acid sequence Met (1) to Ile (1174) of SEQ ID NO. 2. For example, the enzyme may comprise an amino acid sequence having a sequence identity of at least 90% relative to the amino acid sequence Met (1) to Ile (1174) of SEQ ID NO. 2, preferably at least 95%, and even more preferably at least 97.5%. In some instances the enzyme may comprise an amino acid sequence having a sequence identity of at least 99% relative to the amino acid sequence Met (1) to Ile (1174) of SEQ ID NO. 2.

In other preferred embodiments of the invention the amino acid sequence of the enzyme has a sequence identity of at least 80% relative to the amino acid sequence Met (1) to Ile (1174) of SEQ ID NO. 2. For example, the amino acid sequence of the enzyme may have a sequence identity of at least 90% relative to the amino acid sequence Met (1) to Ile (1174) of SEQ ID NO. 2, preferably at least 95%, and even more preferably at least 97.5%. In some instances the amino acid sequence of the enzyme may have a sequence identity of at least 99% relative to the amino acid sequence Met (1) to Ile (1174) of SEQ ID NO. 2.

In some presently preferred embodiments of the invention the enzyme has the amino acid sequence Met (1) to Ile (1174) of SEQ ID NO. 2.

In some embodiments of the invention the enzyme comprises an amino acid sequence having a sequence identity of at least 80% relative to the amino acid sequence Val (33) to Ile (1174) of SEQ ID NO. 2. For example, the enzyme may comprise an amino acid sequence having a sequence identity of at least 90% relative to the amino acid sequence Val (33) to Ile (1174) of SEQ ID NO. 2, preferably at least 95%, and even more preferably at least 97.5%. In some instances the enzyme may comprise an amino acid sequence having a sequence identity of at least 99% relative to the amino acid sequence Val (33) to Ile (1174) of SEQ ID NO. 2.

In other embodiments of the invention the amino acid sequence of the enzyme may have a sequence identity of at least 80% relative to the amino acid sequence Val (33) to Ile (1174) of SEQ ID NO. 2. For example, the amino acid sequence of the enzyme may have a sequence identity of at least 90% relative to the amino acid sequence Val (33) to Ile (1174) of SEQ ID NO. 2, preferably at least 95%, and even more preferably at least 97.5%. In some instances the amino acid sequence of the enzyme may have a sequence identity of at least 99% relative to the amino acid sequence Val (33) to Ile (1174) of SEQ ID NO. 2. Thus, the enzyme may e.g. have the amino acid sequence Val (33) to Ile (1174) of SEQ ID NO. 2.

In some presently preferred embodiments of the invention the enzyme has the amino acid sequence Val (33) to Ile (1174) of SEQ ID NO. 2.

In some embodiments of the invention, the enzyme may e.g. comprise an amino acid sequence having a sequence identity of at least 99% relative to an amino acid sequence shown in Table 1. The enzyme may for example comprise an amino acid sequence shown in Table 1. Alternatively, the amino acid sequence of the enzyme may have a sequence identity of at least 99% relative to an amino acid sequence shown in Table 1. The enzyme may for example have an amino acid sequence shown in Table 1.

TABLE 1
Useful amino acid sequences (AAS).

Position in SEQ ID NO. 2

| AAS No. | From | To |
|---|---|---|
| 1 | 25 | 1122 |
| 2 | 25 | 1132 |
| 3 | 25 | 1142 |
| 4 | 25 | 1152 |
| 5 | 25 | 1162 |
| 6 | 25 | 1167 |
| 7 | 25 | 1168 |
| 8 | 25 | 1169 |
| 9 | 25 | 1170 |
| 10 | 25 | 1171 |
| 11 | 25 | 1172 |
| 12 | 25 | 1173 |
| 13 | 25 | 1174 |
| 14 | 25 | 1175 |
| 15 | 25 | 1176 |
| 16 | 25 | 1177 |
| 17 | 25 | 1178 |
| 18 | 25 | 1179 |
| 19 | 25 | 1180 |
| 20 | 25 | 1181 |
| 21 | 25 | 1186 |
| 22 | 25 | 1196 |
| 23 | 25 | 1206 |
| 24 | 25 | 1216 |
| 25 | 25 | 1226 |
| 26 | 27 | 1122 |
| 27 | 27 | 1132 |
| 28 | 27 | 1142 |
| 29 | 27 | 1152 |
| 30 | 27 | 1162 |
| 31 | 27 | 1167 |
| 32 | 27 | 1168 |
| 33 | 27 | 1169 |
| 34 | 27 | 1170 |
| 35 | 27 | 1171 |
| 36 | 27 | 1172 |
| 37 | 27 | 1173 |
| 38 | 27 | 1174 |
| 39 | 27 | 1175 |
| 40 | 27 | 1176 |
| 41 | 27 | 1177 |
| 42 | 27 | 1178 |
| 43 | 27 | 1179 |
| 44 | 27 | 1180 |
| 45 | 27 | 1181 |
| 46 | 27 | 1186 |
| 47 | 27 | 1196 |
| 48 | 27 | 1206 |
| 49 | 27 | 1216 |
| 50 | 27 | 1226 |
| 51 | 30 | 1122 |
| 52 | 30 | 1132 |
| 53 | 30 | 1142 |
| 54 | 30 | 1152 |
| 55 | 30 | 1162 |
| 56 | 30 | 1167 |
| 57 | 30 | 1168 |
| 58 | 30 | 1169 |
| 59 | 30 | 1170 |
| 60 | 30 | 1171 |
| 61 | 30 | 1172 |
| 62 | 30 | 1173 |
| 63 | 30 | 1174 |
| 64 | 30 | 1175 |
| 65 | 30 | 1176 |
| 66 | 30 | 1177 |
| 67 | 30 | 1178 |
| 68 | 30 | 1179 |
| 69 | 30 | 1180 |
| 70 | 30 | 1181 |
| 71 | 30 | 1186 |
| 72 | 30 | 1196 |
| 73 | 30 | 1206 |
| 74 | 30 | 1216 |
| 75 | 30 | 1226 |

In some embodiments of the invention, the enzyme may e.g. comprise an amino acid sequence having a sequence identity of at least 99% relative to an amino acid sequence shown in Table 2. The enzyme may for example comprise an amino acid sequence shown in Table 2. Alternatively, the amino acid sequence of the enzyme may have a sequence identity of at least 99% relative to an amino acid sequence shown in Table 2. The enzyme may for example have an amino acid sequence shown in Table 2.

TABLE 2
Useful amino acid sequences (AAS).

Position in SEQ ID NO. 2

| AAS No. | From | To |
|---|---|---|
| 76 | 31 | 1122 |
| 77 | 31 | 1132 |
| 78 | 31 | 1142 |
| 79 | 31 | 1152 |
| 80 | 31 | 1162 |
| 81 | 31 | 1167 |
| 82 | 31 | 1168 |
| 83 | 31 | 1169 |
| 84 | 31 | 1170 |
| 85 | 31 | 1171 |
| 86 | 31 | 1172 |
| 87 | 31 | 1173 |
| 88 | 31 | 1174 |
| 89 | 31 | 1175 |
| 90 | 31 | 1176 |
| 91 | 31 | 1177 |
| 92 | 31 | 1178 |
| 93 | 31 | 1179 |
| 94 | 31 | 1180 |
| 95 | 31 | 1181 |
| 96 | 31 | 1186 |
| 97 | 31 | 1196 |
| 98 | 31 | 1206 |
| 99 | 31 | 1216 |
| 100 | 31 | 1226 |
| 101 | 32 | 1122 |
| 102 | 32 | 1132 |
| 103 | 32 | 1142 |
| 104 | 32 | 1152 |
| 105 | 32 | 1162 |
| 106 | 32 | 1167 |
| 107 | 32 | 1168 |
| 108 | 32 | 1169 |
| 109 | 32 | 1170 |
| 110 | 32 | 1171 |
| 111 | 32 | 1172 |
| 112 | 32 | 1173 |
| 113 | 32 | 1174 |
| 114 | 32 | 1175 |
| 115 | 32 | 1176 |
| 116 | 32 | 1177 |
| 117 | 32 | 1178 |
| 118 | 32 | 1179 |
| 119 | 32 | 1180 |
| 120 | 32 | 1181 |
| 121 | 32 | 1186 |
| 122 | 32 | 1196 |
| 123 | 32 | 1206 |
| 124 | 32 | 1216 |
| 125 | 32 | 1226 |
| 126 | 33 | 1122 |
| 127 | 33 | 1132 |
| 128 | 33 | 1142 |
| 129 | 33 | 1152 |
| 130 | 33 | 1162 |
| 131 | 33 | 1167 |
| 132 | 33 | 1168 |
| 133 | 33 | 1169 |
| 134 | 33 | 1170 |
| 135 | 33 | 1171 |
| 136 | 33 | 1172 |
| 137 | 33 | 1173 |
| 138 | 33 | 1174 |
| 139 | 33 | 1175 |
| 140 | 33 | 1176 |
| 141 | 33 | 1177 |
| 142 | 33 | 1178 |
| 143 | 33 | 1179 |
| 144 | 33 | 1180 |
| 145 | 33 | 1181 |
| 146 | 33 | 1186 |
| 147 | 33 | 1196 |
| 148 | 33 | 1206 |
| 149 | 33 | 1216 |
| 150 | 33 | 1226 |

In some embodiments of the invention, the enzyme may e.g. comprise an amino acid sequence having a sequence identity of at least 99% relative to an amino acid sequence shown in Table 3. The enzyme may for example comprise an amino acid sequence shown in Table 3. Alternatively, the amino acid sequence of the enzyme may have a sequence identity of at least 99% relative to an amino acid sequence shown in Table 3. The enzyme may for example have an amino acid sequence shown in Table 3.

TABLE 3
Useful amino acid sequences (AAS).

Position in SEQ ID NO. 2

| AAS No. | From | To |
|---|---|---|
| 151 | 34 | 1122 |
| 152 | 34 | 1132 |
| 153 | 34 | 1142 |
| 154 | 34 | 1152 |
| 155 | 34 | 1162 |
| 156 | 34 | 1167 |
| 157 | 34 | 1168 |
| 158 | 34 | 1169 |
| 159 | 34 | 1170 |
| 160 | 34 | 1171 |
| 161 | 34 | 1172 |
| 162 | 34 | 1173 |
| 163 | 34 | 1174 |
| 164 | 34 | 1175 |
| 165 | 34 | 1176 |
| 166 | 34 | 1177 |
| 167 | 34 | 1178 |
| 168 | 34 | 1179 |
| 169 | 34 | 1180 |
| 170 | 34 | 1181 |
| 171 | 34 | 1186 |
| 172 | 34 | 1196 |
| 173 | 34 | 1206 |
| 174 | 34 | 1216 |
| 175 | 34 | 1226 |
| 176 | 35 | 1122 |
| 177 | 35 | 1132 |
| 178 | 35 | 1142 |
| 179 | 35 | 1152 |
| 180 | 35 | 1162 |
| 181 | 35 | 1167 |
| 182 | 35 | 1168 |
| 183 | 35 | 1169 |
| 184 | 35 | 1170 |
| 185 | 35 | 1171 |
| 186 | 35 | 1172 |
| 187 | 35 | 1173 |
| 188 | 35 | 1174 |
| 189 | 35 | 1175 |
| 190 | 35 | 1176 |
| 191 | 35 | 1177 |
| 192 | 35 | 1178 |
| 193 | 35 | 1179 |
| 194 | 35 | 1180 |
| 195 | 35 | 1181 |
| 196 | 35 | 1186 |
| 197 | 35 | 1196 |
| 198 | 35 | 1206 |
| 199 | 35 | 1216 |
| 200 | 35 | 1226 |
| 201 | 36 | 1122 |
| 202 | 36 | 1132 |
| 203 | 36 | 1142 |
| 204 | 36 | 1152 |
| 205 | 36 | 1162 |
| 206 | 36 | 1167 |
| 207 | 36 | 1168 |
| 208 | 36 | 1169 |
| 209 | 36 | 1170 |
| 210 | 36 | 1171 |
| 211 | 36 | 1172 |
| 212 | 36 | 1173 |
| 213 | 36 | 1174 |
| 214 | 36 | 1175 |
| 215 | 36 | 1176 |
| 216 | 36 | 1177 |
| 217 | 36 | 1178 |
| 218 | 36 | 1179 |
| 219 | 36 | 1180 |
| 220 | 36 | 1181 |
| 221 | 36 | 1186 |
| 222 | 36 | 1196 |
| 223 | 36 | 1206 |
| 224 | 36 | 1216 |
| 225 | 36 | 1226 |

In some embodiments of the invention, the enzyme may e.g. comprise an amino acid sequence having a sequence identity of at least 99% relative to an amino acid sequence shown in Table 4. The enzyme may for example comprise an amino acid sequence shown in Table 4. Alternatively, the amino acid sequence of the enzyme may have a sequence identity of at least 99% relative to an amino acid sequence shown in Table 4. The enzyme may for example have an amino acid sequence shown in Table 4.

TABLE 4
Useful amino acid sequences (AAS).

| | |
|---|---|
| | Position in |
| AAS | SEQ ID NO. 2 |

| No. | From | To |
|---|---|---|
| 226 | 39 | 1122 |
| 227 | 39 | 1132 |
| 228 | 39 | 1142 |
| 229 | 39 | 1152 |
| 230 | 39 | 1162 |
| 231 | 39 | 1167 |
| 232 | 39 | 1168 |
| 233 | 39 | 1169 |
| 234 | 39 | 1170 |
| 235 | 39 | 1171 |
| 236 | 39 | 1172 |
| 237 | 39 | 1173 |
| 238 | 39 | 1174 |
| 239 | 39 | 1175 |
| 240 | 39 | 1176 |
| 241 | 39 | 1177 |
| 242 | 39 | 1178 |
| 243 | 39 | 1179 |
| 244 | 39 | 1180 |
| 245 | 39 | 1181 |
| 246 | 39 | 1186 |
| 247 | 39 | 1196 |
| 248 | 39 | 1206 |
| 249 | 39 | 1216 |
| 250 | 39 | 1226 |
| 251 | 42 | 1122 |
| 252 | 42 | 1132 |
| 253 | 42 | 1142 |
| 254 | 42 | 1152 |
| 255 | 42 | 1162 |
| 256 | 42 | 1167 |
| 257 | 42 | 1168 |
| 258 | 42 | 1169 |
| 259 | 42 | 1170 |
| 260 | 42 | 1171 |
| 261 | 42 | 1172 |
| 262 | 42 | 1173 |
| 263 | 42 | 1174 |
| 264 | 42 | 1175 |
| 265 | 42 | 1176 |
| 266 | 42 | 1177 |
| 267 | 42 | 1178 |
| 268 | 42 | 1179 |
| 269 | 42 | 1180 |
| 270 | 42 | 1181 |
| 271 | 42 | 1186 |
| 272 | 42 | 1196 |
| 273 | 42 | 1206 |
| 274 | 42 | 1216 |
| 275 | 42 | 1226 |

In some embodiments of the invention the enzyme may contain one or more glycosylated amino acid(s). Alternatively, or in addition, the enzyme may contain one or more phosphorylated amino acid(s). Alternatively, none of the amino acids of the enzyme are glycosylated or phosphorylated.

In some preferred embodiments of the invention the enzyme comprises at least two sub-units, each sub-unit consisting of an enzyme as defined above.

The enzyme preferably contacts the mixture and is thereby brought into contact with both the galactosyl acceptor and the galactosyl donor.

In some embodiments of the invention the mixture comprises the enzyme. The enzyme may e.g. be present in the mixture in dissolved form, e.g. as single enzyme molecules or as soluble aggregate of enzyme molecules.

In other embodiments of the invention the enzyme is separate from the mixture, but brought in contact with the galactosyl acceptor and the galactosyl donor by contacting the enzyme with the mixture. For example, enzyme immobilised on a stationary solid phase may be used. Examples of useful stationary solid phases are e.g. a filter, a packed bed of enzyme-containing particles, or similar structures.

Alternatively, the solid phase may e.g. be a free flowing, particulate solid phase, e.g. organic or inorganic beads, forming part of the mixture.

Details relating to the industrial use of enzymes including immobilisation techniques and suitable solid phase types can be found in Buchholz (2005), which is incorporated herein by reference for all purposes.

The enzyme is preferably used in a sufficient activity to obtain an acceptable yield of galacto-oligosaccharides. The optimal activity depends on the specific implementation of the process and can easily be determined by the person skilled in the art.

If a high turn-over of galactosyl donor and a high yield of galacto-oligosaccharide is required, it may be preferred to use the enzyme in a relatively high activity. For example, the activity of the enzyme may be such that the turn-over of the galactosyl donor is at least 0.02 mol/(L*h), preferably at least 0.2 mol/(L*h), and even more preferably at least 2 mol/(L*h).

The enzymatic reaction takes place during step c). As soon as the mixture is exposed to the right conditions, which may be almost immediately when the galactosyl acceptor and the galactosyl donor are brought in contact with the enzyme, the transgalactosylation usually starts, and in some embodiments of the invention steps b) and c) occur simultaneously.

The enzyme is capable of releasing the leaving group of the galactosyl donor and transferring the galactosyl group of the galactosyl donor to the galactosyl acceptor. For example, if the galactosyl donor is lactose, glucose is released and the galactosyl group is transferred to the acceptor. The enzyme acts as catalyst during the enzymatic reaction.

In some preferred embodiments of the invention the enzyme furthermore transfers galactosyl groups to already galactosylated galactosyl acceptors, thereby generating galactosyl acceptors containing two, three or even more galactosyl groups.

The pH of the mixture is preferably near the optimum pH of the enzyme. In some embodiments of the invention the pH of the mixture during step c) is in the range of pH 3-9. For example, the pH of the mixture during step c) may be in the range of pH 4-8, such as in the range of pH 5-7.5.

Similar to the pH, the temperature of the mixture is preferably adjusted to the optimum temperature of the used enzyme. In some embodiments of the invention the temperature during step c) is in the range of 10-80 degrees C. The temperature during step c) may e.g. be in the range of 20-70 degrees C., preferably in the range of 25-60 degrees C., and even more preferably in the range of 30-50 degrees C.

In the context of the present invention, the term “optimum pH of the enzyme” relates to the pH where the enzyme has the highest transgalactosylation activity. Along the same lines, the term “optimum temperature of the enzyme” relates to the temperature where the enzyme has the highest transgalactosylation activity.

The inventors have discovered that the present method surprisingly provides a high yield of galacto-oligosaccharides even though a relatively low concentration of the galactosyl donor is used. The relatively low concentration of galactosyl donor additionally reduces the degree of self-galactosylation of the donor, i.e. when the galactosyl group of a first galactosyl donor is transferred to a second galactosyl donor instead of to a galactosyl acceptor.

In some preferred embodiments of the invention, step c) comprises addition of further galactosyl donor to the mixture. This is particularly preferred when a relatively low concentration of the galactosyl donor is used. By adding more galactosyl donor one avoids the galactosyl donor being depleted in the mixture and the concentration of galactosyl donor may be controlled during the enzymatic reaction.

The addition of further galactosyl donor may involve discrete addition(s) of galactosyl donor, e.g. at least once during the enzymatic reaction. Alternatively, or additionally, the addition of further galactosyl donor may be a continuous addition during the enzymatic reaction. The further galactosyl donor is preferably of the same type as used in step a).

In some preferred embodiments of the invention, the concentration of galactosyl donor of the mixture during step c) is maintained at a concentration in the range of 0.01-1 mol/L, preferably in the range of 0.01-0.5 mol/L, and preferably in the range of 0.03-0.3 mol/L.

For example, the concentration of galactosyl donor of the mixture during step c) may be maintained at a concentration in the range of 0.02-0.1 mol/L.

Step c) may furthermore comprise addition of further galactosyl acceptor. This makes it possible to control the concentration of galactosyl acceptor of the mixture during step c) and e.g. to keep the galactosyl acceptor concentration substantially constant if this is desired.

In order to produce significant amounts of galacto-oligosaccharides, which contain two or three transferred galactosyl groups, the process should consume more galactosyl donor than galactosyl acceptor. Thereby more of the galactosyl acceptors will become galactosylated two or three times. Thus, in some preferred embodiments of the invention the molar ratio between the consumed galactosyl donor and the consumed galactosyl acceptor is at least 1:1, and preferably at least 5:1, and even more preferably at least 10:1.

Often it is required to enrich and/or purify the galacto-oligosaccharides of the composition and reduce the concentration of the galactosyl acceptor, the galactosyl donor and the released leaving group.

Thus, in some preferred embodiments of the invention the method furthermore comprises the step:

d) enriching the galacto-oligosaccharides of the composition of step c).

In the context of the present invention, the term “enriching the galacto-oligosaccharides” relates to increasing the relative amount of the galacto-oligosaccharides of the composition on a dry weight basis. This is typically done by removing some of the other solids of the composition, e.g. the lower saccharides, and optionally also the enzyme, if required.

The enrichment of step d) may for example involve chromatographic separation and/or nanofiltration. Details regarding such processes are described in Walstra et al. (2006) which is incorporated herein by reference for all purposes.

In some embodiments of the invention the enrichment involves that at least 50% (w/w on dry weight basis) of the molecules having a molar weight of at most 200 g/mol are removed from the composition of step c). For example, the enrichment may involve that at least 80% (w/w on dry weight basis) of the molecules having a molar weight of at most 200 g/mol are removed from the composition of step c).

In other embodiments of the invention the enrichment involves that at least 50% (w/w on dry weight basis) of the molecules having a molar weight of at most 350 g/mol are removed from the composition of step c). For example, the enrichment may involve that at least 80% (w/w on dry weight basis) of the molecules having a molar weight of at most 350 g/mol are removed from the composition of step c).

As an alternative, or in addition, to the enrichment it may be preferred that step d) comprises one or more processes which increase the concentration of the galacto-oligosaccharides in the composition. Examples of useful concentration steps are e.g. reverse osmosis, evaporation, and/or spray-drying.

The galacto-oligosaccharide-containing composition provided by the method may for example be in the form of a dry powder or in the form of a syrup.

The production of a dry powder typically requires one or more process steps, such as concentrating, evaporating, and/or spray-drying. Thus, in some preferred embodiments of the invention the step d) furthermore involves concentrating, evaporating, and/or spray-drying the composition in liquid form to obtain the composition in powder form. It is particularly preferred to spray-dry the liquid composition of step d) to obtain a powdered composition. Step d) may for example comprise the enrichment step followed by concentration step, e.g. nanofiltration, reverse osmosis, or evaporation, followed by a spray-drying step. Alternatively, step d) may comprise the concentration step followed by an enrichment step, followed by a spray-drying step. Concentrating the galacto-oligosaccharides of the composition prior to the enrichment may make the subsequent enrichment process more cost-efficient.

Efficient spray-drying may require addition of one or more auxiliary agent(s), such as maltodextrin, milk protein, caseinate, whey protein concentrate, and/or skimmed-milk powder.

The present process may e.g. be implemented as a batch process. The present process may alternatively be implemented as a fed-batch process. The present process may alternatively be implemented as a continuous process.

The present process may furthermore involve recirculation of enzyme and/or unused galactosyl acceptor back to the mixture. The recirculation may e.g. form part of step d). For example, step d) may involve separating galactosyl acceptor and/or the enzyme from the galacto-oligosaccharide-containing composition and recirculating galactosyl acceptor and/or enzyme to step a), or c). In the case of a batch process or a fed-batch process, the galactosyl acceptor and/or the enzyme may be recirculated to the mixture of the next batch.

In the case of a continuous process, the galactosyl acceptor may be recirculated back to part of the process line corresponding to step a) or step c). The enzyme may be recirculated back to part of the process line corresponding to step b) or step c).

It should be noted that the details and features related to steps a) and b) need not relate to the actual start of a production process, but should at least occur sometime during the process. However, in some embodiments of the invention the concentration of the galactosyl donor is kept within the range described in step a) during the entire duration of step c).

If the method is implemented as a batch or feed batch process, step a) preferably pertains to the composition of the mixture when the synthesis starts. If the method is a continuous process, step a) preferably pertains to the composition of the mixture during the synthesis under steady-state operation.

It may be perceived as desirable that the level of galactosylated galactosyl donor is kept as low as possible, as galactosylated galactosyl donor may be perceived as an undesired impurity, which is tricky to separate from the galactosylated galactosyl acceptor. In some preferred embodiments of the invention, the mixture of step a) contains at most 0.5 mol/L galactosylated galactosyl donor. The galactosylated galactosyl donor may for example contain at most 0.1 mol/L galactosylated galactosyl donor. Even more preferably the galactosylated galactosyl donor contains at most 0.01 mol/L galactosylated galactosyl donor, and preferably substantially no galactosylated galactosyl donor.

Yet an aspect of the invention relates to a composition comprising galacto-oligosaccharides, which composition is obtainable by the method as defined herein.

A further aspect of the invention is a galacto-oligosaccharide-containing composition, e.g. the above-mentioned composition, said galacto-oligosaccharide-containing composition comprising:
a first galacto-oligosaccharide having the general formula Gal-X,
a second galacto-oligosaccharide having the stoichiometric formula (Gal)₂X, such as e.g. the general formula Gal-Gal-X,
a third galacto-oligosaccharide having the stoichiometric formula (Gal)₃X, such as e.g. the general formula Gal-Gal-Gal-X, and
   wherein X is a glycosyl group, which is not lactosyl or glucosyl.

The galacto-oligosaccharide-containing composition described herein may for example be a food ingredient.

As described above, “X” or “—X” is preferably a glycosyl group of one of the galactosyl acceptors mentioned herein.

In some embodiments of the invention “X” or “—X” is a glycosyl group of a monosaccharide, which is not glucose. In other embodiments of the invention “−X” is a glycosyl group of a disaccharide, which is not lactose.

In some preferred embodiments of the invention “X” or “—X” is a fucosyl group. In other preferred embodiments of the invention “X” or “—X” is a galactosyl group.

In some preferred embodiments of the invention the galacto-oligosaccharide-containing composition has a molar ratio between:
the total amount of the galacto-oligosaccharides Gal-X, (Gal)₂X, and (Gal)₃X, and
the total amount of the galacto-oligosaccharides Gal-Glc, Gal₂Glc, Gal₃Glc
   of at least 5:95. For example, the above-mentioned molar ratio may be at least 1:4, preferably at least 1:1, and even more preferably at least 2:1. It may even be preferred that the above-mentioned molar ratio is at least 5:1, preferably at least 10:1, and even more preferably at least 20:1.

In other preferred embodiments of the invention the galacto-oligosaccharide-containing composition has a molar ratio between:
the total amount of the galacto-oligosaccharides Gal-X, Gal-Gal-X, and Gal-Gal-Gal-X, and
the total amount of the galacto-oligosaccharides Gal-Glc, Gal-Gal-Glc, Gal-Gal-Gal-Glc
   of at least 5:95. For example, the above-mentioned molar ratio may be at least 1:4, preferably at least 1:1, and even more preferably at least 2:1. It may even be preferred that the above-mentioned molar ratio is at least 5:1, preferably at least 10:1, and even more preferably at least 20:1.

It is even possible that the galacto-oligosaccharide-containing composition does not contain any galacto-oligosaccharides of the formula Gal-Glc, Gal-Gal-Glc, and Gal-Gal-Gal-Glc at all.

In some embodiments of the invention the galacto-oligosaccharide-containing composition has a molar ratio between the first galacto-oligosaccharide, the second galacto-oligosaccharide, and the third galacto-oligosaccharide in the range of 50-99:1-45:0.5-25.

In other embodiments of the invention the galacto-oligosaccharide-containing composition has a molar ratio between the first galacto-oligosaccharide, the second galacto-oligosaccharide, and the third galacto-oligosaccharide in the range of 20-45:20-45:20-45.

In further embodiments of the invention the galacto-oligosaccharide-containing composition has a molar ratio between the first galacto-oligosaccharide, the second galacto-oligosaccharide, and the third galacto-oligosaccharide in the range of 0.5-25:1-45:50-98.

In some preferred embodiments of the invention the galacto-oligosaccharide-containing composition comprises a total amount of the first galacto-oligosaccharide, second galacto-oligosaccharide, and third galacto-oligosaccharide of at least 10% by weight relative to the total weight of the galacto-oligosaccharide-containing composition. For example, the galacto-oligosaccharide-containing composition may comprise a total amount of the first galacto-oligosaccharide, second galacto-oligosaccharide, and third galacto-oligosaccharide of at least 20% by weight relative to the total weight of the galacto-oligosaccharide-containing composition, preferably at least 30% by weight, even more preferably at least 40% relative to the total weight of the galacto-oligosaccharide-containing composition.

Even higher levels of the first, second, and third galacto-oligosaccharides may be preferred. Thus, in some preferred embodiments of the invention the galacto-oligosaccharide-containing composition comprises a total amount of the first galacto-oligosaccharide, second galacto-oligosaccharide, and third galacto-oligosaccharide of at least 50% by weight relative to the total weight of the galacto-oligosaccharide-containing composition. For example, the galacto-oligosaccharide-containing composition may comprise a total amount of the first galacto-oligosaccharide, second galacto-oligosaccharide, and third galacto-oligosaccharide of at least 60% by weight relative to the total weight of the galacto-oligosaccharide-containing composition, preferably least 70% by weight, even more preferably at least 80% relative to the total weight of the galacto-oligosaccharide-containing composition.

Yet an aspect of the invention relates to a food product comprising the galacto-oligosaccharide-containing composition described herein.

In some embodiments of the invention the food product is a functional food product such as infant formula or a product for clinical nutrition.

In other embodiments of the invention the food product is a baked product, e.g. comprising baked dough, such as bread or similar products.

In further embodiments of the invention the food product is a dairy product, e.g. a fresh dairy product such as milk, or a fermented dairy product such as yoghurt.

In still further embodiments of the invention the food product is a pet food product.

It should be noted that embodiments and features described in the context of one of the aspects of the present invention also apply to the other aspects of the invention.

The invention will now be described in further details in the following non-limiting examples.

### EXAMPLES

### Example 1

### Preparation of the Enzyme

A working volume of 750 mL fermentation medium was inoculated with a 2 mL starter-culture of Lysogeny broth (LB) medium with 100 mg/L ampicillin with an OD₆₀₀ of 3.0 grown for 12 hours. The fermentation was performed in EC medium containing 2% (w/v) yeast extract, 2% (w/v) soy peptone, 1% (w/v) glucose and 100 mg/L ampicillin. The E. coli strain expressing OLGA347 β-galactosidase was prepared as described earlier (Jorgensen et al., U.S. Pat. No. 6,555,348 B2, Examples 1 and 2). The fermentor was from Applikon with glass dished bottom vessels with a total volume of 2 L and equipped with two Rushton impellers. During the fermentation, pH was maintained at pH 6.5 by appropriate addition of 2 M NaOH and 2 M H₃PO₄ and temperature was controlled at 37 degrees C. Oxygen was supplied by bubbling with air at a rate of 1-2 L/min, and pO₂ was maintained at 30% by increasing the agitation rate. Growth was followed by off-line OD₆₀₀ readings. The culture was harvested by centrifugation after approximately 10 h of growth at an OD₆₀₀ value of 29.7. The 650 mL culture supernatant was stored at -20 degrees C. The periplasmic proteins were isolated from the cell pellet by osmotic shock by resuspending the cell pellet in 200 mL sucrose buffer (30 mM Tris-HCl, 40% sucrose, 2 mM EDTA, pH 7.5) and incubating for 10 min at room temperature. After centrifugation, the supernatant was discarded and the pellet resuspended in 200 mL of cold water. 83 μL of a saturated MgCl₂ solution was added, and the supernatant containing the periplasmic proteins were collected by a centrifugation step. The periplasmic fraction was filter sterilized through a 0.2 μm Millipak 40 filter and stored at −20 degrees C.

The pβ-galactosidase activity of the 200 mL periplasmic fraction and the 650 mL culture supernatant was determined using o-nitrophenyl-β-D-galactopyranoside (OPNG) as a substrate according to protocol (J. Sambrook and D. W. Russell, Molecular Cloning—A laboratory manual, 3^{rd} edition (2001), pp. 17.48-17.51). The majority of the activity was found in the periplasmic fraction (525 units, corresponding to 98%).

### Example 2

### Determination of the T-value of a beta-galactosidase Enzyme

The T-value of a beta-galactosidase enzyme is determined according to the assay and formula given below.

### Assay:

Prepare 3.3 mL enzyme solution consisting of the beta-galactosidase enzyme to be tested, 10 mM sodium citrate, 1 mM magnesium citrate, 1 mM calcium-citrate, Milli-Q water (Millipore, USA), and having a pH of 6.5. The enzyme solution should contain the beta-galactosidase enzyme in an amount sufficient to use 33% (w/w) of the added lactose in 1 hour under the present assay condition. The temperature of the enzyme solution should be 37 degrees C.

At time=T₀ 82.5 mg lactose monohydrate (for biochemistry, Merck Germany) is added to and mixed with, the enzyme solution, and the mixture is subsequently incubated at 37 degrees C. for 4 hours. Precisely 1 hour after T₀ a 100 μL sample is collected and is diluted 1:5 with Milli-Q water and inactivated by heating to 85° C. for 10 min. The inactivated mixture is kept at −20 degrees C. until the characterization.

### Characterisation:

The determination of the amount (in mol) of produced galactose and the amount of used lactose (in mol) may be performed using any suitable analysis technique. For example, the diluted mixture may be analyzed by HPLC according to the method described by Richmond et al. (1982) and Simms et al. (1994). Other useful analysis techniques are described in El Razzi (2002).

Another example of a suitable analysis technique is ISO 5765-2:2002 (IDF 79-2:2002) “Dried milk, dried ice-mixes and processed cheese—Determination of lactose content—Part 2: Enzymatic method utilizing the galactose moiety of the lactose”.

### Calculation of the T-value:

The T-value is calculated according to the following formula using the data obtained from the characterization of the diluted mixture of the assay:

$$T \text{-} value = \frac{amount of produced galactose ( in mol )}{amount of used lactose ( in mol )}$$

### Example—T-value of the OLGA347 Enzyme:

The above-mentioned assay was performed using the OLGA347 enzyme of Example 1.

The diluted mixture obtained from the assay was analyzed with respect to converted (i.e. used) lactose and generated galactose via analytical HPLC. The HPLC apparatus was from Waters and equipped with a differential refractometer (RI-detector) and a BioRad Aminex HPX-87C column (300×7.8 mm, 125-0055). Elution of saccharides was performed isocratically with 0.05 g/L CaAcetate, a flow rate of 0.3 mL/min. and an injection volume of 20 μL.

The obtained data was appropriately baseline corrected by automated software, peaks were individually identified and integrated. Quantification was performed by using external standards of lactose monohydrate (for biochemistry, Merck, Germany), D-(+)-glucose monohydrate (for biochemistry, Merck Eurolab, France), and D-(+)-galactose (≧99%, Sigma-Aldrich, Italy).

The conversion of lactose and the formation of galactose to each time T was calculated from the quantified data. At time T=1 h 29% of the lactose in the collected 100 μL sample had been converted, which corresponds to 2.3 μmol lactose. At time T=10.5 μmol galactose had been formed in the collected 100 μL sample. The T-value can therefore be calculated to 0.5 μmol/2.3 μmol=0.2.

The diluted mixture obtained from the assay was also analyzed with respect to converted (i.e. used) lactose and generated galactose via the enzymatic method ISO 5765-2. A Boehringer Mannheim Lactose/D-Galactose test-kit from R-Biopharm (Cat. No. 10 176 303 035) was used and the test performed according to protocol. The enzymatic method confirmed a T-value of the OLGA347-enzyme of 0.2.

### Example

### T-value of Conventional Lactase Enzyme

The above-mentioned assay was performed using the commercially available conventional lactase enzyme Lactozym Pure 2600L (Novozymes, Denmark). The diluted mixture obtained from the assay was analyzed as described for the OLGA347 enzyme. Tri- and tetra-saccharides were not present in detectable amounts and equal amounts of glucose and galactose were seen. The corresponding T-value is 1.

The T-values of commercially available beta-galactosidase from Escherichia coli (Product number: G6008, Sigma-Aldrich, Germany) and Aspergillus oryzae (Product number: G5160, Sigma-Aldrich, Germany) have also been determined, and both enzymes have a T-value of approx. 1.

### Example 3

### Synthesis of L-fucosyl-containing Hetero-galacto-oligosaccharides By Sequential Addition of Donor Molecules

700 mg L-(−)-Fucose (99%, Sigma-Aldrich, Slovakia) and 20 mg lactose monohydrate (for biochemistry, Merck, Germany) was dissolved in 5 mL buffer (10 mM Na-Citrat, 20 mM Na₂HPO₄, pH 6.5) and maintained at a temperature of 37 degrees C. 2 mL OLGA347 enzyme, prepared as in Example 1, was added. This time is defined as T=0. During a 6 h period 20 mg lactose monohydrate was added with 30 min. intervals. 100 μL samples were acquired at times T=0, 1, 2, 3, 4, 5, and 7 h. Sample acquisition and characterization was done as in example 2. Mass spectrometry analysis was performed with an Agilent 1100 API-ES LC/MSD Quadropole scanning masses between 100 and 1000 amu (gas temperature: 350° C., drying gas flow: 13.0 L/min, nebulizer pressure: 60 psig). The detected ions arise from complexation between the analyte and sodium cations from the solution, resulting in detected masses of M(Na⁺)=M+23 Da.

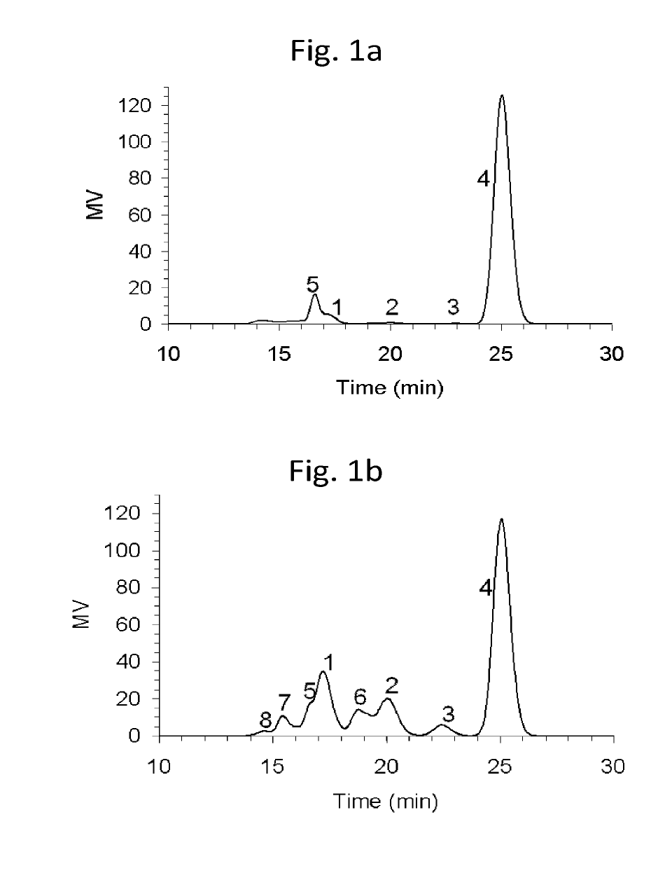

HPLC chromatograms from T=0 h and T=7 h are presented in FIG. 1a and FIG. 1b, respectively. The peaks have been identified by MS (data not shown) and are labeled in the figures. Legend: 1=lactose, 2=glucose, 3=galactose, 4=L-fucose, 5=sucrose, 6=Gal-Fuc disaccharides, 7=Gal-Gal-Fuc & Gal-Gal-Glc trisaccharides, 8=Gal-Gal-Gal-Fuc & Gal-Gal-Gal-Glc tetrasaccharides. In the figures it can be seen that the concentration of glucose and galactose increases with glucose being approximately 4 times more abundant than galactose. This is in accordance with the assumption that galactose is being used as donor molecule for the formation of galacto-oligosaccharides and the remaining glucose being released into solution. The concentration of lactose increases due to continuing addition of lactose. This peak also includes allolactose and Gal-Gal disaccharides formed in the enzymatic reaction. Furthermore, Gal-Fuc disaccharides, Gal-Gal-Glc and Gal-Gal-Fuc trisaccharides and Gal-Gal-Gal-Glc and Gal-Gal-Gal-Fuc tetrasaccharides are formed.

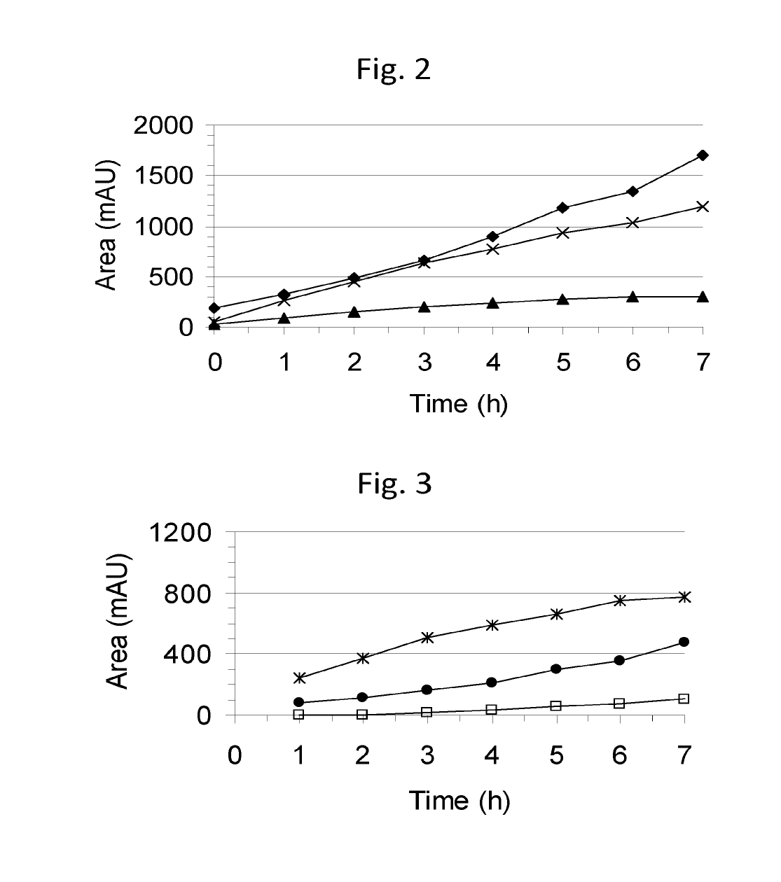

Plots of the calculated peak area as a function of time for lactose, glucose and galactose are presented in FIG. 2. Legend: diamond=lactose, cross=glucose, triangle=galactose. It is seen that the concentration of lactose, allolactose and Gal-Gal disaccharides increases and that the concentration of glucose increases linearly as glucose is being released into the solution during the enzymatic reaction. The concentration of galactose is low compared to glucose and the concentration increases only slowly. This indicates that almost all galactose provided from the cleavage of lactose has been used to form galacto-oligosaccharides. Furthermore, the concentration of L-fucose decreases during the course of the experiment, as L-fucose is being used as acceptor molecule in the enzymatic reaction to form galacto-oligosaccharides (data not shown).

FIG. 3 shows a plot of the calculated peak area as a function of time for the products from the enzymatic reaction. Legend: star =Gal-Fuc disaccharide, circle =Gal-Gal-Glc & Gal-Gal-Fuc trisaccharide, hollow square =Gal-Gal-Gal-Glc & Gal-Gal-Gal-Fuc tetrasaccharide. Both Gal-Fuc disaccharides and tri- and tetrasaccharides are formed. The trisaccharides are of the form Gal-Gal-Glc and Gal-Gal-Fuc, and the tetrasaccharides are of the form Gal-Gal-Gal-Glc and Gal-Gal-Gal-Fuc.

The concentration of Gal-Fuc disaccharides increases linearly and shows a tendency of reaching a plateau from T=6 h to T=7 h. The concentration of Gal-Gal-Glc and Gal-Gal-Fuc trisaccharides increases linearly and shows a tendency of exponential increase from T=4 h. The concentration of the Gal-Gal-Gal-Glc and Gal-Gal-Gal-Fuc tetrasaccharides increases linearly.

The amounts (w/w of total carbohydrate) of L-fucose-containing galacto-oligosaccharides are estimated based on HPLC and MS data at T=7 h: Gal-Fuc=8%, Gal-Gal-Fuc=3%, Gal-Gal-Gal-Fuc=1%. In all, L-fucose-containing galacto-oligosaccharides constitute 12% after a reaction time of T=7 h. Upon removal of free L-fucose by chromatography, the calculated amount of L-fucose-containing galacto-oligosaccharides is 28%.

### Example 4

### Synthesis of D-fucosyl-containing Hetero-galacto-oligosaccharides

110 mg D-(+)-Fucose (≧98%, Sigma-Aldrich, Slovakia) and 55 mg lactose monohydrate (for biochemistry, Merck, Germany) was dissolved in 1 mL buffer (10 mM Na-Citrat, 20 mM Na₂HPO₄, pH 6.5). 100 μL OLGA347 enzyme, which was prepared as in Example 1, was added. This time is defined as T=0. 100 μL samples were acquired at times T=0, 2, 4, 6, and 22 h. Sample acquisition and HPLC characterization was done as in example 2. MS characterization was done as in example 3.

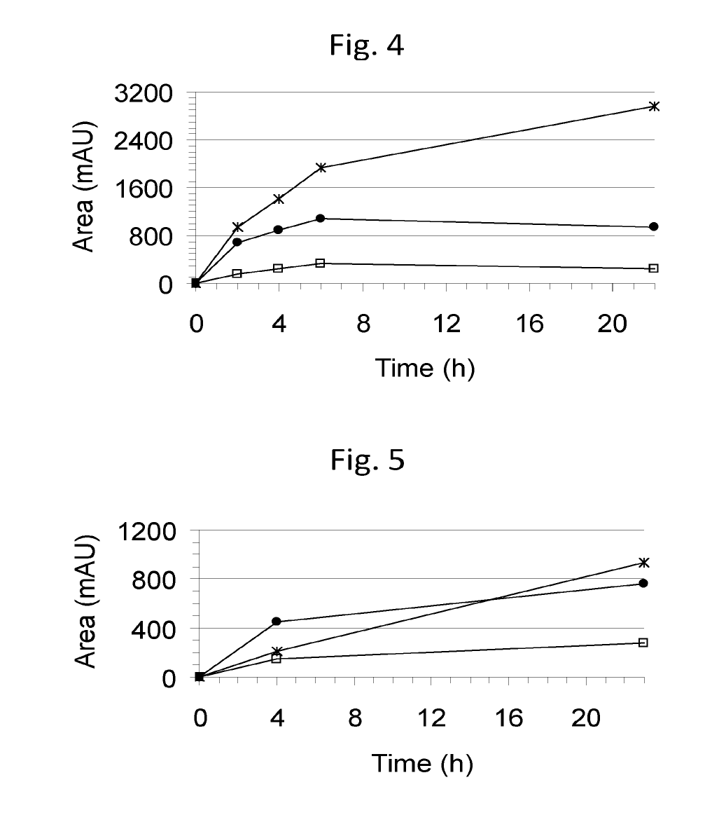

FIG. 4 shows a plot of the calculated peak area as a function of time for the products from the enzymatic reaction. Legend: star=Gal-Fuc disaccharide, circle=Gal-Gal-Glc & Gal-Gal-Fuc trisaccharide, hollow square=Gal-Gal-Gal-Glc & Gal-Gal-Gal-Fuc tetrasaccharide. Both Gal-Fuc disaccharides and tri- and tetrasaccharides are formed. The trisaccharides are of the form Gal-Gal-Glc and Gal-Gal-Fuc, and the tetrasaccharides are of the form Gal-Gal-Gal-Glc and Gal-Gal-Gal-Fuc.

The concentration of Gal-Fuc disaccharides shows the largest increase from T=0 to T=6 h. From T=6 to T=22 h the rate of increase is lower. The concentration of Gal-Gal-Glc and Gal-Gal-Fuc trisaccharides shows the second largest increase from T=0 to T=6 h. From T=6 to T=22 h the concentration drops to the level of T=4 h. The concentration of the Gal-Gal-Gal-Glc and Gal-Gal-Gal-Fuc tetrasaccharides increases from T=0 to T=6 h. From T=6 to T=22 h the concentration drops to the level of T=4 h.

The amounts (w/w of total carbohydrate) of D-fucose-containing galacto-oligosaccharides are estimated based on HPLC and MS data from T=22 h. Gal-Fuc=20%. Gal-Gal-Fuc=6, and Gal-Gal-Gal-Fuc=1%. In all, D-fucose-containing galacto-oligosaccharides constitute 27% after a reaction time of T=22 h. Upon removal of free D-fucose by chromatography, the calculated amount of D-fucose-containing galacto-oligosaccharides is 55%.

### Example 5

### Synthesis of N-acetyl Galactosamine-containing Hetero-galacto-oliqosaccharides

The experiment was conducted as in example 4, only with 110 mg N-Acetyl-D-galactosamine (GalNAc) (98%, Sigma-Aldrich, Germany) as acceptor molecule. 100 μL samples were acquired at times T=0, 4, and 23 h. Sample acquisition and HPLC characterization was done as in example 2. MS characterization was done as in example 3.

FIG. 5 shows a plot of the calculated peak area as a function of time for the products from the enzymatic reaction. Legend: star =Gal-GalNAc disaccharide, circle=Gal-Gal-Glc & Gal-Gal-GalNAc trisaccharide, hollow square =Gal-Gal-Gal-Glc & Gal-Gal-Gal-GaINAc tetrasaccharide. Both Gal-GaINAc disaccharides and tri- and tetrasaccharides are formed. The trisaccharides are of the form Gal-Gal-Glc and Gal-Gal-GaINAc, and the tetrasaccharides are of the form Gal-Gal-Gal-Glc and Gal-Gal-Gal-GalNAc.

The concentration of Gal-GalNAc disaccharides increases linearly from T=0 to T=23 h, and is the most abundant galacto-oligosaccharide at T=23 h. The concentration of Gal-Gal-Glc and Gal-Gal-GalNAc trisaccharides shows the largest increase from T=0 to T=4 h. From T=4 to T=23 h the rate of increase in concentration is lower and almost reaches the Gal-GalNAc level at T=23 h. The concentration of the Gal-Gal-Gal-Glc and Gal-Gal-Gal-Fuc tetrasaccharides increases from T=0 to T=23 h with the largest increase from T=0 to T=4 h. The amounts (w/w of total carbohydrate) of N-acetyl-galactosamine-containing galacto-oligosaccharides are estimated based on HPLC and MS data from T=23 h. Gal-GalNAc=8%, Gal-Gal-GalNAc=5%, and Gal-Gal-Gal-GalNAc=2%. In all, GalNAC-containing galacto-oligosaccharides constitute 15% after a reaction time of T=23 h. Upon removal of free GaINAc by chromatography, the calculated amount of N-acetyl-galactosamine-containing galacto-oligosaccharides is 40%.

### Example 6

### Synthesis of Xylosyl-containing Hetero-galacto-oligosaccharides

The experiment was conducted as in example 4, only with 110 mg D-(+)-Xylose (≧99%, Sigma-Aldrich, USA) as acceptor molecule. 100 μL samples were acquired at times T=0, 5, and 23 h. Sample acquisition and HPLC characterization was done as in example 2. MS characterization was done as in example 3.

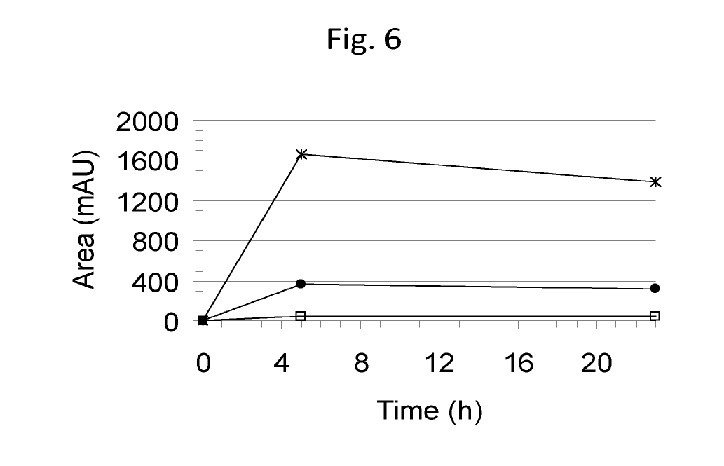

FIG. 6 shows a plot of the calculated peak area as a function of time for the products from the enzymatic reaction. Legend: star=Gal-Xyl disaccharide, circle=Gal-Gal-Glc & Gal-Gal-Xyl trisaccharide, hollow square=Gal-Gal-Gal-Glc & Gal-Gal-Gal-Xyl tetrasaccharide. Both Gal-Xyl disaccharides and tri- and tetrasaccharides are formed. The trisaccharides are of the form Gal-Gal-Glc and Gal-Gal-Xyl, and the tetrasaccharides are of the form Gal-Gal-Gal-Glc and Gal-Gal-Gal-Xyl.

The concentration of Gal-Xyl disaccharides shows the largest increase from T=0 to T=5 h. From T=5 to T=23 h the concentration drops to the level of T=4 h. The concentration of Gal-Gal-Glc and Gal-Gal-Xyl trisaccharides shows the second largest increase from T=0 to T=5 h. From T=5 to T=23 h the concentration drops to the level of T=4 h. The concentration of the Gal-Gal-Gal-Glc and Gal-Gal-Gal-Fuc tetrasaccharides increases from T=0 to T=5 h. From T=5 to T=23 h the concentration does not change at an observable level.

The amounts (w/w of total carbohydrate) of xylosyl-containing galacto-oligosaccharides are estimated based on HPLC and MS data from T=23 h. Gal-Xyl=15%. Gal-Gal-Xyl=3%. Gal-Gal-Gal-Xyl=0.5%. In all, xylosyl-containing galacto-oligosaccharides constitute 18.5% after a reaction time of T=23 h. Upon removal of free Xylose by chromatography, the calculated amount of xylosyl-containing galacto-oligosaccharides is 41%.

### REFERENCES

Buchholz (2005) “Biocatalysts and Enzyme technology”, Klaus Buchholz et al., ISBN-10: 3-527-30497-5, 2005, Wiley VCH Verlag GmbH
Franck (2002) “Technological functionality of inulin and oligofructose”, A. Franck, British Journal of Nutrition (2002), 87, Suppl. 2, S287-S291
Yun (1996) “Fructooligosaccharides-Occurrence, preparation, and application”, J. W. Yun, Enzyme and Microbial Technology 19: 107-117, 1996
Kunz (2000) “Oligosaccharides in human milk: Structural, functional and metabolic aspects”, Kunz et al., Ann. Rev. Nutr. 2000. 20:699-722
Simms et al. (1994) Simms, P. J.; Hicks, K. B.; Haines, R. M.; Hotchkiss, A. T. and Osman, S. F.; (1994) Separations of lactose, lactobionic and lactobionolactose by high performance liquid chromatography. J. of Chromatography, 667, 67-73.
Richmond et al. (1982) Richmond, M. L.; Barfuss, D. L.; Harte, B. R.; Gray, J. I. and Stine, C. M.; (1982) Separation of Carbohydrates in Dairy Products by High Performance Liquid Chromatography, J. of Dairy Science, 65 (8), 1394-1400.
El Razzi (2002) “Carbohydrate Analysis by Modern Chromatography and Electrophoresis”, volume 66, Journal of Chromatography Library, Elsevier Science, 2002, ISBN-10:0444500618
Walstra et al. (2006) “Dairy science and technology”, Walstra et al., CRC Press, Second edition, 2006

1-15. (canceled)

16. A method of producing a composition comprising one or more galacto-oligosaccharide(s), the method comprising the steps of:
a) providing a mixture comprising:
i) a galactosyl donor comprising a galactosyl group bound to a leaving group, which galactosyl donor has a molar weight of at most 350 g/mol;
ii) a galactosyl acceptor which is different from the galactosyl donor;
wherein said galactosyl acceptor is a saccharide or a sugar-alcohol, and
wherein the molar ratio between the galactosyl acceptor and the galactosyl donor is at least 1:10, and
wherein the mixture comprises at least 0.05 mol/L of the galactosyl acceptor;
b) providing an enzyme having beta-galactosidase activity and having a T-value of at most 0.9;
c) contacting the mixture of step a) with the enzyme; and
d) allowing the enzyme to release the leaving group of the galactosyl donor and transfer the galactosyl group of the galactosyl donor to the galactosyl acceptor, thus forming the galacto-oligosaccharide, and thereby obtaining the composition comprising the galacto-oligosaccharide.

17. The method of claim 16, wherein the leaving group of the galactosyl donor is a glycosyl group.

18. The method of claim 16, wherein the enzyme is selected from the group consisting of:
a) an amino acid sequence having a sequence identity of at least 80% relative to the amino acid sequence of SEQ ID NO. 2;
b) an amino acid sequence having a sequence identity of at least 80% relative to the amino acid sequence Met (1) to Ile (1174) of SEQ ID NO. 2;
c) an amino acid sequence having a sequence identity of at least 80% relative to the amino acid sequence Met (1) to Gly (1752) of SEQ ID NO. 2; and
d) an amino acid sequence having a sequence identity of at least 80% relative to the amino acid sequence Val (33) to Ile (1174) of SEQ ID NO. 2.

19. The method of claim 16, wherein the enzyme comprises an amino acid sequence having a sequence identity of at least 90% relative to the amino acid sequence Val (33) to Ile (1174) of SEQ ID NO. 2.

20. The method of claim 16, wherein the enzyme has an amino acid sequence having a sequence identity of at least 90% relative to the amino acid sequence Val (33) to Ile (1174) of SEQ ID NO. 2.

21. The method of claim 16, wherein the enzyme has the amino acid sequence Val (33) to Ile (1174) of SEQ ID NO. 2.

22. The method of claim 16, wherein step c) comprises addition of further galactosyl donor.

23. The method of claim 16, wherein the concentration of galactosyl donor of the mixture during step c) is maintained at a concentration in the range of 0.01-1 mol/L.

24. The method of claim 16, further comprising the step of: enriching the galacto-oligosaccharide of the composition of step c).

25. The method of claim 24, wherein the enrichment comprises chromatographic separation and/or nanofiltration.

26. A galacto-oligosaccharide-containing composition produced by the method of claim 16.

27. A galacto-oligosaccharide-containing composition comprising:
a) a first galacto-oligosaccharide having the general formula Gal-X,
b) a second galacto-oligosaccharide having the general formula Gal₂X, and
c) a third galacto-oligosaccharide having the general formula Gal₃X; wherein X is a glycosyl group, which is not lactosyl or glucosyl, and wherein the molar ratio between:
i) the total amount of the galacto-oligosaccharides Gal-X, Gal₂X, and Gal₃X, and
ii) the total amount of the galacto-oligosaccharides Gal-Glc, Gal₂Glc, Gal₃Glc is at least 5:95.

28. The galacto-oligosaccharide-containing composition of claim 27, wherein the molar ratio between the first galacto-oligosaccharide, the second galacto-oligosaccharide, and the third galacto-oligosaccharide comprises:
50-99 parts of the first galacto-oligosaccharide to
1-45 parts of the second galacto-oligosaccharide to
0.5-25 parts of the third galacto-oligosaccharide (50-99:1-45:0.5-25).

29. The galacto-oligosaccharide-containing composition of claim 28, wherein the total amount of the first galacto-oligosaccharide, second galacto-oligosaccharide, and third galacto-oligosaccharide of is at least 10% by weight, relative to the total weight of the galacto-oligosaccharide-containing composition.

30. A food product comprising the galacto-oligosaccharide-containing composition of claim 26.

31. A galacto-oligosaccharide-containing composition produced by the method of claim 27.

32. A galacto-oligosaccharide-containing composition produced by the method of claim 28.

33. A galacto-oligosaccharide-containing composition produced by the method of claim 29.


SEQUENCE LISTING (2 sequences)

SEQ ID NO: 1 - DNA, 5509 bp, Bifidobacterium bifidum
  CDS (212)..(5470)
atgcgttgcgttgcgatttttccggccctgtatgggggatacaggattggcgatggcgacacgccgtttttgttaatggcatttacatgaaatacaggtaatgagatatcattctcatgatcaccgtgtggatatcgcattggtgcgtatacactaacagcaacagagcggcgcggcaggcgctcgtggattcaatgaagaaggaacgtttatggcagttcgcagacttggtggccgcatcgtggctttcgccgccacagtggccttgtcaataccgttagggttgttaacaaattcagcgtgggcggtcgaggacgccacccgatccgactccaccacgcagatgagctccacgccggaggtggtctactccagcgccgtggattccaagcagaatcgcacctcggatttcgacgccaactggaagttcatgctgtccgattccgtgcaggcgcaggatccggcgttcgacgattcggcctggcagcaggtcgacctgccgcatgactacagcatcacgcagaagtattcgcagagcaacgaggccgaaagcgcataccttcccggcggcaccggctggtaccgcaagtccttcaccatcgaccgggacctcgccggcaagcgcatcgccatcaacttcgacggcgtgtacatgaacgccaccgtctggttcaacggcgtcaagctcggcacccatccgtacggctactcgccgttctccttcgacctgaccggcaacgccaagttcggtggggagaacaccatcgtcgtcaaggtcgagaacaggctgccgtccagccgctggtactccggctccggcatctaccgcgacgtcaccctcaccgtcaccgacggcgtgcacgtcggcaataacggcgtggccatcaagaccccgagcctcgccacccaaaacggcggcgacgtgacgatgaacctcaccaccaaggtcgccaacgacaccgaggccgcggcgaacatcaccctcaagcagaccgtgttccccaagggaggcaagaccgacgccgccatcggcaccgtcaccaccgcatccaagtccatcgcggccggtgccagcgcggacgtgacctccacgatcaccgccgcttcgcccaagctgtggagcatcaagaacccgaacctgtacaccgtgcgcaccgaagtgctcaacggcggcaaggtgctcgacacttacgacaccgaatatggcttccgctggaccggcttcgatgcgaccagcggtttctcgctcaacggcgagaaagtcaagctcaagggcgtctcaatgcatcatgaccagggatcgctcggcgcggtcgccaaccgccgcgccatcgagcgccaggtcgagattctccagaagatgggcgtcaactcgatccgcaccacgcacaaccccgcagccaaggcgctgattgacgtctgcaacgagaagggcgtcctcgtggtcgaagaggtcttcgacatgtggaaccggtcgaagaacggcaacaccgaggattacggcaagtggttcggccaggccatcgccggtgacaacgccgtcctgggtggcgacaaggacgagacctgggccaagttcgacctgaccagcaccatcaaccgtgacaggaacgccccgtccgtcatcatgtggtcgctcggcaacgagatgatggaaggcatcagcggcagcgtctcgggcttcccggctacctccgccaagctggtcgcatggacgaaggccgcggacagcacccgcccgatgacctacggcgacaacaagatcaaggccaactggaacgagtcgaacaccatgggcgacaacctgaccgccaacggcggcgtggtcggcaccaactactccgacggcgcgaactacgacaagatccgcacgacccacccctcatgggccatctatggttccgagacggcgtccgccatcaacagccgaggcatctacaaccgcaccaccggcggcgcccagtcaagcgacaagcagctgaccagctatgacaattccgcagtcggctggggcgccgtcgccagctccgcctggtacgacgtggtccagcgcgatttcgtcgccggcacatacgtgtggaccggcttcgactacctcggcgaacccaccccgtggaacggcaccggctccggcgccgtgggctccttggccgtcgccgaagaactcgtacttcggcatcgtcgacaccgcaggcttcccgaagacacctattacttctatcagagccagtggaacgacgacgtgcacacgctgcacatcctccccgcatggaacgagaacgtcgtcgccaagggctccggcaacaacgtgccggtcgtcgtctacaccgacgcggccaaggtcaagctgtacttcacaccgaagggcagtaccgaaaagcgactgatcggagagaagtccttcaccaagaagaccaccgcggccggatacacctatcaggtctacgagggctccgacaaggactccaccgcccacaagaacatgtacctgacctggaacgtgccgtgggccgagggcaccatctccgccgaagcatacgacgagaacaacaggctgatccccgaggggtccaccgagggcaacgcgtcggtgaccaccaccggcaaggccgcgaagcttaaagccgatgccgaccgcaagacgatcaccgcggacggcaaggacctgtcgtacatcgaggtcgacgtgaccgacgccaacggccatatcgtccccgatgccgccaaccgcgtcaccttcgacgtcaagggcgccggcaaactggtcggcgtcgacaacggcagctcgccggatcacgactcctatcaggccgacaaccgcaaggcgttcagcggcaaggtgctcgccatcgtccagtccaccaaggaggcgggcgagatcaccgtcaccgccaaggccgacggtctgcaatcatccacagtgaagatcgccaccaccgccgtccccggcaccagcaccgagaagacggtccgcagcttctactactcgcgcaactactacgtcaagaccggcaacaagccgattctgccgagtgatgtcgaggtgcgctactccgacggcacgtcggaccgtcagaacgtcacatgggacgcagtcagcgacgaccagatcgccaaggccggttcgttcagcgtggccggcacggtcgccgggcagaagatctccgtgcgcgtgacgatgatcgacgagatcggtgcgctgctcaactattcggccagcacaccggtcggcacgcccgccgtgctgcctggctcgcgtccggccgtgctgcccgacggcaccgtgaccagcgcgaacttcgccgtccactggaccaagcccgccgacaccgtgtacaacacggccggcaccgtcaaggtccccggcaccgccaccgtcttcggcaaggagttcaaggtcaccgcgacgattcgcgtgcagcggtcgcaggtcaccatcggcagcagcgtctccggcaatgcgctgcgcctgactcagaacatccccgccgacaagcagtccgacacgctggacgccatcaaggacggctccacgaccgtcgacgccaataccggcggcggcgcgaacccgtcagcatggaccaactgggcgtactcgaaggccggccacaacaccgccgagatcaccttcgagtacgcgaccgagcagcagctcggccagattgtcatgtacttcttccgcgacagcaacgcggtgaggttccccgacgccggcaagacgaagatccagatctccgcggacggcaagaactggacggatctcgctgccacggagaccatcgcggcccaggagtcgtccgaccgagtcaagccgtacacctatgacttcgctccggtgggagccacgttcgtcaaggtcacggtcaccaacgccgacaccacaacccccagcggcgtggtctgcgccggcctgaccgagatcgagctgaagaccgcgaccagcaagttcgtcacgaacacgtccgccgcgctctcgtcgctgacagtgaacggcacgaaggtctccgactccgtgctcgccgccggctcctacaacacgcccgcgatcatcgcggacgtcaaagccgagggcgaaggcaacgccagcgtcaccgtgctgcccgcgcacgacaacgtgatccgcgtgatcaccgagtccgaggaccacgtcacgcgcaagaccttcaccatcaacctgggcacggagcaggaattccccgcagactccgatgaacgcgactacccggccgccgacatgacggtcaccgtgggcagcgaacagacgtccggcaccgcgaccgaaggcccgaagaaattcgcggtcgacggcaacaccagcacgtactggcattccaactggacgcccaccaccgtgaacgacctgtggatcgccttcgagctccagaaacccaccaagctcgacgcgctgcgctacctgccgcgccccgcgggcagcaagaacggctccgtcaccgaatacaaggttcaggtcagcgatgacggcaccaactggaccgacgcgggctccggcacatggaccaccgattacggctggaagctcgccgagttcaatcagccggtgaccaccaagcacgtgcggctcaaggccgtccacacctatgcggattccggcaacgacaagttcatgtccgcctccgaaatccgcctgcgcaaggccgtcgacaccaccgacatcagcggcgcgaccgtgaccgtgcccgccaagctgaccgtcgaccgggtggacgccgaccatcccgccaccttcgccacgaaggacgtgacggtgacgttgggcgacgccacgctgcgctacggcgtggactacctgctcgactacgcgggcaacaccgccgtcggcaaggccacggtgaccgtgcgcggcatcgacaagtactccggcaccgtcgccaagacgttcaccatcgaactgaagaacgccccggcgccggaaccgacgctgacctcggtgagcgtcaagaccaagccttccaagctgacctatgtggtcggcgacgcgttcgacccggcaggactggtgctgcagcacgacagacaggccgatcgccccccacagccacttgttggagaacaggccgacgaacgcggactgacgtgcggaacgcgatgcgatcgcgttgaacagctgcgcaaacacgagaatcgtgaagcccatcgtacgggcctcgatcatctggaattcgtgggtgccgccgatggagcggtcggtgaacaggccaccttcaaggtgcatgtccatgccgatcaaggtgacggccgccatgatgatgccgatgaacgcgatatcgatccacatgtccctgtcgatcacgcggtcggtgagcttgcgcgggctgcgtgccatcacgtcatcggtctgcgggtcgacacccatcgcctcaaggcatccggcttccagatccccgccgacgacatggccgagatcgaccgcatcaccggcttccaccgcttcgagcgccacgtcggctgacgtgattgggcttccccgctgtctggtgccggctcgcga

SEQ ID NO: 2 - protein, 1752 aa, Bifidobacterium bifidum
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSLAVAEELVLRHRRHRRLPEDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG